(19) United States
(12) Patent Application Publication
(10) Pub. No.: US 2005/0048144 A1
(43) Pub. Date: Mar. 3, 2005

(54) Herb extract-based cosmeceutical cream for controlling the blood sugar level of diabetes and methods for making it

(76) Inventors: Xiao-Qing Han, Naperville, IL (US); MingXia Liu, Naperville, IL (US)

Correspondence Address: Xiao-Qing Han, 1464 Larsen Court, Naperville, IL, 60563, US

(21) Appl. No.: 10/651,194

(22) Filed: Aug. 29, 2003

(51) Int. Cl. A61K 35/78

(52) U.S. Cl. 424/732; 424/741; 424/752; 424/757; 424/762; 424/764; 424/754

(57) ABSTRACT

The current invention provides an herb extract based anti-diabetic cosmeceutical cream product for controlling the blood sugar levels of non-insulin dependent diabetes and methods for making the product. The invention includes a blood sugar lowering product containing hypocaemics ingredients of anti-diabetic herb extracts, a skin care cream base, and hypocaemics drugs. The invention describes details in the herb extracts that have the function of hypocaemics and improvement on other symptoms resulting from the increased blood sugar levels. The invention provides formulation skills and a formula for making an herb extract based anti-diabetic cream product that can penetrate animal and especially human skin to deliver active anti-diabetic elements. Finally, the invention includes methods for producing an herb extract based anti-diabetic cream product.

### FIELD OF THE INVENTION

The present invention relates to a functional cosmetic cream product with skin penetration property to deliver biologically active components to control blood sugar levels of diabetes and methods for making the product. More specifically, the present invention relates to a product of herb extract based cream with biological functions in lowering blood sugar level of type II diabetes and improving the sensitivity of beta-cell response to insulin.

### BACKGROUND

Diabetes is classified into two types. Type I is known as immune-mediated diabetes (formerly called insulin-dependent diabetes). Type I diabetes destroys the cells in the pancreas that produce insulin, usually leading to a total failure to produce insulin. People with Type 1 diabetes must give themselves at least one shot of insulin every day. In Type II diabetes, or non-insulin-dependent diabetes mellitus, the body either makes insufficient amounts of insulin or is unable to use it. Type II diabetes often develops slowly. Most people who get it have increased thirst and an increased need to urinate. Many also feel edgy, tired and sick to their stomach. Some people have an increased appetite, but they lose weight. Other signs include repeated or hard-to-heal infections of the skin, gums, vagina or bladder; blurred vision; tingling or loss of feeling in the hands or feet; and dry, itchy skin. Type II diabetes accounts for 90 to 95 percent of all cases of diagnosed diabetes. Type II diabetes is known to cause problems with the kidneys, legs and feet, eyes, heart, nerves and blood flow.

More than 135 million people worldwide have diabetes and every year 2.8 million people die from the disease. This number has increased 15% in the last ten years and is expected to double by 2005. Currently there is no known cure for diabetes. The Centers for Disease Control and Prevention refer to diabetes as the “epidemic of our time.” In the United States, about 16 million people suffer from diabetes mellitus, although only half of these individuals have been diagnosed. Every year, about 650,000 people learn they have the disease. Diabetes mellitus is the seventh leading cause of all deaths and the sixth leading cause of all disease-caused deaths. Every day in the United States alone, diabetes will blind 75 people, cause 80 people to suffer kidney failure, and will result in 50 leg or foot amputations.

In both Type 1 and Type 2 diabetes, blood sugar, blood pressure, and blood fats must be well controlled to prevent possible development of blindness, kidney failure, and heart disease. Also, tiny blood vessels in the body may become blocked—a dangerous complication. When blood vessels of the eye are affected, it can result in retinopathy, the breakdown of the lining at the back of the eye. The treatment for Type 2 diabetes, therefore, generally focuses on the reduction of blood glucose levels. Increased blood glucose, which is caused by defective insulin secretion and decreased insulin sensitivity, is the hallmark of diabetes.

Although there are several blood-sugar-lowering drugs available currently on the market, all of them are either designed for oral intake or injection, which are inconvenient and often cause side effects that would be dangerous to elderly patents. All of the oral hypoglycemic drugs have the potential to interact with other medications and if the result is hypoglycemia or hyperglycaemia the consequences can be serious. Oral hypoglycaemic drugs available currently on the market include: sulfonylureas, biguanides, alpha glucosidase inhibitors, and thiazolidinediones.

Sulfonylurea is one of a group of oral hypo-glycaemic drugs derived from a sulphonamide, which are used in the treatment of noninsulin-dependent diabetes mellitus. They act by stimulating the islet cells in the pancreas to produce more insulin. The group includes chlorpropamide, gliclazide tolazamide, Glimepiride, glibenclamide, and tobutamide. All drugs in this class are partially or totally metabolised by the liver. These drugs stimulate insulin secretion and hence may cause hypos and also stimulate weight gain. Drugs with shorter half-lives are preferable (eg. gliclazide and glipizide). Chlorpropamide has a very long half-life, and Glibenclamide also has a long half-life. Therefore, they all need careful monitoring due to the risk of hypoglycaemic episodes. These drugs are often used in combination with Metformin if the patient is overweight.

Metformin is currently the only drug used that is of biguanide class, which reduces blood sugar levels and is used to treat noninsulin-dependent diabetes. Metformin is particularly useful in obese diabetics as it helps suppress appetite and diminishes hepatic glucose excretion. Because it does not stimulate insulin release, Metformin does not cause hypos. However, care must be taken in patients with renal or hepatic impairment, or patients may develop an acute illness such as lactic acidosis and the drug should be ceased until the illness has passed. Metformin is administered by mouth and may cause loss of appetite and minor digestive upsets.

Sulfonylurea and biguanide have been the mainstay of drug treatment for type 2 diabetes. Recently, there are three new types of drugs available, which include; acarbose (an alphaglucosidase inhibitor), repaglinide (a meglitinide) and the glitazones' (thiazolidinediones).

Acarbose is a competitive inhibitor of the alpha glucosidase enzymes that are responsible for the breakdown of complex carbohydrate into their sugar components ready for absorption. The complex carbohydrate is therefore absorbed further down the small intestine, leading to a slower rise in the post-prandial blood sugar levels and improving post-prandial glycaemic control. The drug may be used either alone or in combination with other diabetic therapies including insulin, and may reduce HbA1cs by anywhere up to 1% over a period of months. The carbohydrate load that enters the large bowel however often leads to intolerable GIT side effects.

The thiazolidinediones were designed to increase insulin sensitivity in Type 2 diabetics, of which Troglitazone is the most commonly used agent. Side effects of thiazolidinediones include liver and bone marrow toxicity.

In fact, all oral hypo-glycaemic drugs have side effects that would be dangerous to patients (especially elderly patents). Side effects of oral administration of hypoglycaemic drugs could also occur due to the drug interactions with the concomitant administration of other foods and/or drugs. Furthermore, the side effects of orally administrated and/or injected drugs are irreversible, and can hardly be dismissed or removed once the injection or oral treatment is completed. The irreversibility of side effects from oral administration and/or injection of drugs, therefore, increase the dangerous to patients. For some elderly patients, swallowing an entire tablet could also be a challenge.

Numerous strategies have been developed to achieve the goal of controlling blood glucose at a relatively constant level in diabetic patients.

U.S. Pat. No. 6,608,038 teaches art on methods and compositions for treatment of diabetes and related conditions via gene therapy. The methods include gene therapy based administration of a therapeutically effective amount of vectors encoding the following: glucokinase regulatory protein alone or co-administered with glucokinase or with metabolism modifying proteins; glucokinase co-administered with metabolism modifying proteins; or glucokinase regulatory protein co-administered with glucokinase in combination with metabolism modifying proteins, to a diabetic patient.

U.S. Pat. No. 6,586,438 teaches art on a low dose antidiabetic pharmaceutical formulation, especially adapted for treating Type II diabetes in drug naive patients, which includes a combination of metformin (employed in a reduced amount (less than 800 mg metformin per day) compared to that employed in generally accepted medical practice) and at least one other antidiabetic agent such as a sulfonyl urea.

U.S. Pat. No. 6,576,270 teaches art on herbal composition and medicament against diabetes mellitus type II. The herbal composition according the invention is dried product processed for oral consumption.

U.S. Pat. No. 6,562,379 discloses an adult-onset diabetes treatment method by administering to the mammal Momordica lectin or pokeweed mitogen chloroform precipitatable fraction.

U.S. Pat. No. 6,551,627 teaches art on medicinal herbal compounds for the prevention and treatment of diabetes. The hypoglycemic effective composition comprising extracts from Pterocarpus marsupium, Morus alba, Orthosiphon aristatus, Opiophogon japonicus, Anemarrhena asphodeloides, or Trichosanthis kirilowii, in combination with extracts from Rosa rugosa and/or Commelina communis, and a pharmaceutically acceptable carrier. The carrier, according to the invention, is a base of berries or fruit, a base of vegetable soup or bouillon, a soya-milk drink, or a nutritive supplement.

U.S. Pat. No. 6,339,076 teaches art on making therapeutic food compositions for treatment of diabetic patients to diminish fluctuations in blood sugar levels and to prevent hypoglycemic episodes.

U.S. Pat. No. 6,337,075 teaches a method for treating hyperinsulinemic type 2 diabetes by local administration of a neurotoxin, such as a botulinum toxin, into the pancreas, thereby reducing insulin secretion from a B cell, and a method for treating hypoinsulinemic type 2 diabetes by local administration of a neurotoxin, such as a botulinum toxin, into a sympathetic ganglion, thereby reducing an inhibitory effect upon insulin secretion.

U.S. Pat. No. 6,174,543 disclosed an antidiabetic external skin application composition containing glibenclamide and benzyl alcohol and optionally a nonionic surfactant.

U.S. Pat. No. 6,147,108 teaches a method for treating or preventing type II diabetes mellitus comprising administering an effective amount of a gastrointestinal lipase inhibitor, such as, tetrahydrolipstatin.

U.S. Pat. No. 6,093,403 discloses an herbal formulation for treating and preventing non-insulin dependent diabetes mellitus (NIDDM) and other sugar imbalances. The herbal formulation is added to the diet of a person suffering from NIDDM in a lectin mixture containing the herbal formulation.

U.S. Pat. No. 6,042,834 discloses an herbal composition for the treatment of diabetes, comprising dried powdered: seeds of Trigonella foenum-graecum, seeds of Nigella sativa, leaves of Origanum vulgare, sap of Rosmarinus officinalis, beans of Lupinus termis, black leaves of Lawsonia inermis, and seeds of Foeniculum vulgare. The herbal composition may be administered in non-encapsulated powder form or in capsule form.

U.S. Pat. No. 5,266,561 discloses compounds and methods for blocking the effects of diabetes-associated peptide, or “amylin”, a hormone found in the amyloid masses of Type 2 diabetics.

None of the above references suggests employing herb-based cream product that can penetrate human skin and deliver anti-diabetic components for lowering blood sugar levels of patients.

Thus, there remains a need for additional anti-diabetic products without requiring the injection or oral administration of drugs. There is a need for developing safer and easier methods of lowering blood sugar levels of diabetes. There is also a need for developing a convenient and yet effective method of lowering the blood sugar levels without requiring the injection or oral administration of drugs. Furthermore, there remains a need for developing methods of lowering the blood sugar levels of diabetes, whereby side effects can be minimized, or dismissed by patients (removable) when they sense the side effects. Additionally, there remains a need for developing anti-diabetic products that can be utilized externally are more convenient to carry, and that provide skin care functions to diabetic patients. More importantly, there remains a need for developing herb-extract-based anti-diabetic skin application products that can release symptoms after a long term of use. Most importantly, there remains a need for developing the formula and methods for making the product with the advantages described above. The current invention provides a novel product concept with detailed approaches to fulfilling the above needs.

### SUMMARY OF THE INVENTION

The object of the present invention is to eliminate the above-mentioned problems still associated with prior arts and to provide an external skin application product capable of lowering the blood sugar levels of non-insulin dependent diabetics. The present invention, as a result of extensive studies and research, has found that an external skin application product containing the effective ingredients of herb extracts together with currently in using oral anti-diabetic drugs can be formulated and processed into a product with excellent skin absorption and blood sugar lowering functions.

According to the present invention, biologically active compounds with blood sugar lowering capacity and other functions that could in the long term improve physiological situations of diabetes, could be incorporated into a well designed skin care product to make a novel multifunctional skin care product. In the present invention, the biologically active blood sugar lowering compounds are extracts of traditional herb medicines. The current invention reveals that combining the orally administrated anti-diabetic drugs currently on the market with extracts of traditional anti-diabetic herb medicines provides several synergic advantages including: more effective blood sugar lowering capacity, reducing thirst, increasing energy, etc.

All diabetic patients have an increased level of blood glucose, and some patients also have secondary effects from the diabetes, such as poor circulation, platelet adhesion and aggregation, excess free radicals, shortages of antioxidants, heart disease, cataracts, and liver and kidney problems. Most of these are caused, at least in part, by too high levels of glucose. Excess glucose can be toxic to cells due to the formation of advanced glycation end products (AGE) and free radicals such as O₂.—, .OH, which contribute to diabetic complications. Therefore, the ideal drug for diabetes treatment would be one that not only controlled the glucose levels, but also dealt with some of the complications and secondary effects of diabetes.

Although the drugs of modern medicine (i.e. insulin and oral hypo-glycaemics) are the main regulators of gross plasma glucose levels, herbal medicines have a lot to offer in treating and preventing the complications described above. Therefore, the invention includes the selection of traditional anti-diabetic herb medicines to be formulated with orally administrated anti-diabetic drugs. The invention formula includes additives for preventing oxidation of biologically active ingredients. The invention includes stabilized products with improved storage characteristics that are oxidation resistant and Theologically stable. The invention also includes methods for incorporating the extract of traditional anti-diabetic herb medicines into the product. Finally, the invention includes methods for making the product.

In one aspect, the present invention is an external skin application product capable of lowering the blood sugar levels of non-insulin dependent diabetes, wherein the external skin application product contains extracts of traditional anti-diabetic herb medicines selected from the group consisting of Vaccinium myrtillus, Crataegus oxyacantha/C. monogyna, Baical Skullcap, Ginkgo biloba, Gymnema sylvestra, Fenugreek, Galega officinalis, and onion. In one embodiment, the traditional anti-diabetic herb medicine is Galega officinalis (Goat's Rue). In another embodiment, the traditional anti-diabetic herb medicine is Trigonella foenum-graecum (Fenugreek). In another embodiment, the traditional anti-diabetic herb medicine is Gymnema sylvestra. In another embodiment, the traditional anti-diabetic herb medicine is Ginkgo biloba. In another embodiment, the traditional anti-diabetic herb medicine is Baical Skullcap. In another embodiment, the traditional anti-diabetic herb medicine is Crataegus oxyacantha/C. monogyna (Hawthorn). In another embodiment, the traditional anti-diabetic herb medicine is Vaccinium myrtillus (Bilberry). In another embodiment, the traditional anti-diabetic herb medicine is ginseng. In another embodiment, the traditional anti-diabetic herb medicine is onion.

In one aspect, the present invention is an external skin application product consisting of a blood sugar lowering anti-diabetic herb extract selected from the group consisting of traditional medicines including Vaccinium myrtillus, Crataegus oxyacantha/C. monogyna, Baical Skullcap, Ginkgo biloba, Gymnema sylvestra, Fenugreek, Galega officinalis, and onion, the said extract being present in an amount effective for the purpose of lowering glucose levels and other improvement to the diabetic patients in long term use. In one embodiment of this aspect of the invention, the traditional anti-diabetic herb medicine ingredient subject to extraction is a mix of water soluble and solvent extracted parts of a subject herb medicine. In one embodiment, the ingredient mixed with water soluble and solvent soluble parts of extracts is ginseng extract. In another embodiment, the ingredient mixed with water soluble and solvent soluble parts of extracts is onion extract. In another embodiment, the ingredient mixed with water soluble and solvent soluble parts of extracts is Vaccinium myrtillus extract. In another embodiment, the ingredient mixed with water soluble and solvent soluble parts of extracts is Crataegus oxyacantha/C. monogyna extract. In another embodiment, the ingredient mixed with water soluble and solvent soluble parts of extracts is Baical Skullcap extract. In another embodiment, the ingredient mixed with water soluble and solvent soluble parts of extracts is Ginkgo biloba extract. In another embodiment, the ingredient mixed with water soluble and solvent soluble parts of extracts is Gymnema sylvestra extract. In another embodiment, the ingredient mixed with water soluble and solvent soluble parts of extracts is Fenugreek extract. In another embodiment, the ingredient mixed with water soluble and solvent soluble parts of extracts is Galega officinalis extract.

In one embodiment of this aspect of the invention, the product ingredient subject to hypo-glycaemics is a cosmetic ingredient and the product is a skin care cream. In another embodiment, the product ingredient subject to hypo-glycaemics is a medical reagent component and the product is a blood lowering medical reagent. In another embodiment, the product ingredient subject to hypo-glycaemics is a pharmaceutical and the product is an anti-diabetic pharmaceutical product.

In another aspect, the current invention is a hypo-glycaemics therapeutic compound for treating non-insulin dependent diabetic disease, wherein the therapeutic compound comprises: a group of oral hypo-glycaemic sulfonylurea drugs derived from a sulphonamide, an oral hypo-glycaemic drug of biguanide class, thiazolidinediones, meglitinide, and an alphaglucosidase inhibitor, wherein the hypo-glycaemics therapeutic compound has hypo-glycaemics activity, and wherein the hypo-glycaemics therapeutic compound is present in an amount effective for the purpose of lowering glucose levels and other improvement to the diabetic patients in long term of use. In one embodiment of this aspect of the invention directed to a compound for treating a disease involving increased blood glucose levels, the hypo-glycaemics therapeutic compound consists essentially of the sulfonylurea drugs derived from a sulphonamide comprising chlorpropamide, gliclazide tolazamide, Glimepiride, glibenclamide, and tobutamide. In another embodiment, the hypo-glycaemics therapeutic compound consists essentially of the drug of biguanide class comprising only Metformin currently. Sulfonylurea drugs can be used in combination with Metformin. In another embodiment, the hypo-glycaemics therapeutic compound consists essentially of the drug of an alphaglucosidase inhibitor. In another embodiment, the hypo-glycaemics therapeutic compound consists essentially of the drug of thiazolidinediones. In another embodiment, the hypo-glycaemics therapeutic compound consists essentially of the drug of a meglitinide.

In another aspect, the current invention provides a method for treating a condition associated with hyperglycaemics in a subject; said method comprises administering to the subject an amount of an hypo-glycaemics therapeutic compound as described herein. In one embodiment of this aspect of the invention directed to a method for treating a condition associated with hyperglycaemics, the subject is a mammal, including a human. In another embodiment of this aspect of the current invention, the condition associated with hyperglycaemics is a non-insulin dependent diabetic disease.

### BRIEF DESCRIPTION OF THE DRAWINGS

FIG. 1 is a flow diagram of one embodiment of a method for making the blood sugar lowering skin care cream product.

The following nomenclature is used:

HYDROPHILIC SYSTEM: A solution mix comprises substances that are all water-soluble at formulated levels of concentration at room temperature or higher.

HYDROPHOBIC STSTEM: A solution mix comprises substances that are all water-insoluble at formulated levels of concentration at room temperature or higher, but can be dissolved in solvent(s) selected at certain temperatures.

HYPOGLYCAEMICS: Substances that have the capacity of lowering glucose levels in vivo when administrated.

HERB EXTRACTS: A mix of water soluble and solvent extracted parts of a subject herb medicine used in the invention.

### DESCRIPTION OF THE PREFERRED EMBODIMENTS

Blood sugar lowering skin care creams. In one aspect, the current invention is a blood sugar lowering skin care cream comprising an hypocaemics ingredients of anti-diabetic herb extracts, a skin care cream base, and hypocaemics drugs and correlated solvents for dissolving them, wherein the blood sugar lowering skin care cream has hypocaemic activity. In one embodiment, the extract of traditional anti-diabetic herb medicines is selected from the group consisting of Vaccinium myrtillus, Crataegus oxyacantha/C. monogyna, Baical Skullcap, Ginkgo biloba, Gymnema sylvestra, Fenugreek, Galega officinalis, and onion, wherein the quantity of the extract added to the product is in the range of 0.2% to 20% by weight. In one embodiment, the method for treating a condition associated with hyperglycaemics in a subject; said method comprises administering to the subject an amount of a hypo-glycaemics therapeutic compound as described herein. In one embodiment of this aspect of the invention directed to a method for treating a condition associated with hyperglycaemics, the subject is a mammal, including a human. In another embodiment of this aspect of the current invention, the condition associated with hyperglycaemics is a non-insulin dependent diabetic disease.

This aspect of the present invention typically includes a pharmaceutically acceptable carrier—a skin penetration system that delivers hypocaemics into animal body to lower blood sugar levels caused by diabetic disease. The term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, excipient, adjuvant, or vehicle with which the therapeutic is administered through non-oral approaches. Such pharmaceutical carriers can be a mixture of a sterile emulsion system containing hydrophilic and hydrophobic components such as water and oils, including those of petroleum, animal, vegetable, or synthetic origin, such as peanut oil, soybean oil, mineral oil, sesame oil, and the like. Water is a preferred carrier when the pharmaceutical composition is in hydrophilic nature. Suitable pharmaceutical excipients include glucose, lactose, sucrose, starch, malt, rice, gelatin, flour, silica gel, chalk, sodium stearate, talc, sodium chloride, glycerol monostearate, dried skim milk, glycerol, propylene, glycol, ethanol, water, and the like. The pharmaceutically acceptable carrier in current invention also contains minor amounts of wetting or emulsifying agents, and pH buffering agents. Such therapeutic blood sugar lowering cream will contain a therapeutically effective amount of the active hypocaemics ingredient, preferably in purified form, together with a suitable amount of carrier so as to provide proper administration to the patient. The formulation should suit the mode of administration.

Other cosmetic compositions in the blood sugar lowering skin care cream. The blood sugar lowering skin care cream composition, according to the present invention, may contain other known components of cosmetic compositions, as long as the objects of the present invention can be accomplished. Examples of such components are waxes, carnauba wax, silicones, hydrocarbons such as liquid paraffin, oil and fats such as Macadamia nut oil, lanolin, higher alcohols, humectants (such as polyethylene glycol, propylene glycol), cholesterol, higher fatty acids, carboxylic acid salts, short-chain soluble collagen, antioxidants, lower alcohols such as ethanol, antibiotic agent such as benzoic acid, amino acids, organic acids, vitamins such as, for example, vitamin A and the derivatives thereof, vitamin Bs such as vitamin B6 hydrochloride, vitamin B6, vitamin B12, vitamin C such as ascorbic acid, ascorbic acid sulfate esters (salts), vitamin Es, vitamin Ds, vitamin H, anionic surfactants such as sodium laurate, potassium laurate, sodium laurylsulfate, sodium palmitate, acylmethyl taurine salts, triethanolamine alkyl sulfate; cationic surfactants such as laurylamine oxide, stearyl trimethylammonium chloride, benzalkonium chloride; amphoteric surfactant; neutralizing agents; sequestering agent; fragrances; etc.

Other drug components in the blood sugar lowering skin care cream The blood sugar lowering skin care cream composition according to the present invention may contain other known components of anti-diabetic drug compositions, as long as the objects of the present invention can be accomplished. Some anti-diabetic drugs can have synergic effect and improve hypocaemic activity of the present invention. Examples of such components are drugs or agents such as chlorpropamide, glibenclamide, gliclazide, glimepiride, glipizide, tolbutamide, glycyrrhizic acid (salt), glycyrrhetic acid, nicotinic amide, pantothenyl ethyl ether, allantoin, hinokitiol, bisabold, eucalyptone, thymol, inositol, tranexamic acid, arbutin, cepharanthine, saphora flavescens, malva sylvestris, and thiazolidinediones.

In one embodiment, the above therapeutic components can be delivered in a vesicle, in particular a liposome (see Treat et al., in Liposomes in the Therapy of Infectious Disease and Cancer, Lopez-Berestein and Fidler (eds.), Liss, N.Y., pp. 353-365 (1989); Langer, “New methods of drug delivery,” Science 249:1527 (1990)) and incorporated into the herb-extract based cream to improve hypocaemic activity of the present invention.

In yet another embodiment, the therapeutic drug components in the anti-diabetic cream can be delivered in a controlled release system. In one embodiment, a pump may be used (see Langer, (1990); Sefton, “Implantable pumps,” Crit. Rev. Biomed. Eng. 14: 201 (1987); and Saudek et al., “A preliminary trial of the programmable implantable medication system for insulin delivery,” N. Engl. J. Med. 321:574 (1989)). In another embodiment, polymeric materials can be used (see Ranger et al., Macromol. Sci. Rev. Macromol. Chem. 23:61 (1983); During et al., “Controlled release of dopamine from a polymeric brain implant: in vivo characterization,” Ann. Neurol. 25:351 (1989). Other controlled release systems discussed in the review by Langer et al. (1990) can also be used.

The amount of the blood sugar lowering skin care cream of the invention which will be effective in the treatment of type-II diabetics will depend on the nature of the disorder or condition, as well as the stage of the disorder or condition. Effective amounts can be determined by standard clinical techniques. The precise dose to be employed in the formulation should be decided according to the judgment of the health care practitioner and each patient's circumstances. Effective doses may be extrapolated from dose-response curves derived from in vitro or animal model test systems. However, suitable dosage ranges for the blood sugar lowering skin care cream are generally about 200 to about 2000 micrograms.

The following examples describe and illustrate the methods and compositions of the invention. These examples are intended to be merely illustrative of the present invention, and not limiting thereof in either scope or spirit. Unless indicated otherwise, all percentages are by weight. Those skilled in the art will readily understand that variations of the materials, conditions, and processes described in these examples can be used.

### EXAMPLE 1

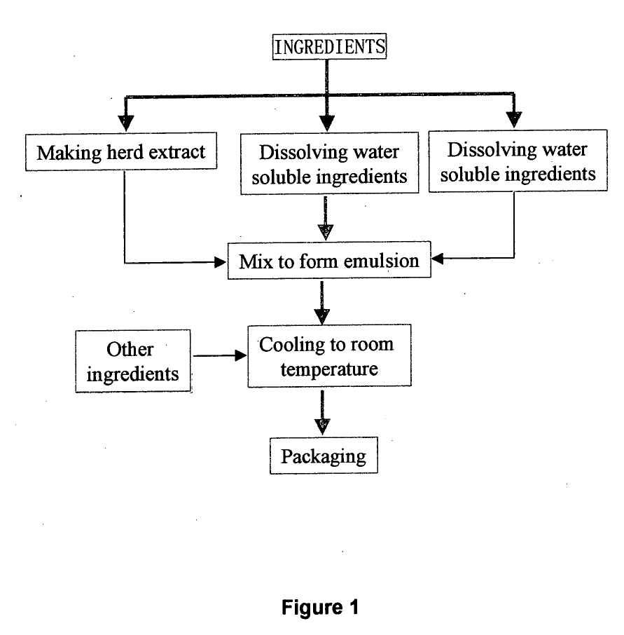

A) Weigh ingredients as indicated in the following formula and processed as shown in FIG. 1:

| | |
|---|---|
| 1) Glimepiride | 1.82% |
| 2) Cepharanthine | 0.4% |
| 3) Vitamin E | 0.3% |
| 4) Glycerol monostearate | 4.2% |
| 5) PEG-12 | 2.6% |
| 6) Ethanol | 1.8% |
| 7) Cetyl alcohol | 7.2% |
| 8) Bio-Soft | 0.04% |
| 9) Wax | 12% |
| 10) Tn-glycerol stearate | 0.3% |
| 11) Ginkgo biloba extract | 5.8% |
| 12) Fenugreek extract | 4.4% |
| 13) Sodium benzoate | 0.08% |
| 14) Sodium sulfite | 0.8% |
| 15) Sodium chloride | 0.7% |
| 16) Vitamin C | 0.6% |
| 17) Tween-20 | 0.2% |
| 18) Fragrant | 0.5% |
| 19) Add water to | 100% |

B) Mix ingredients 1-10 together and heat to 80° C.
C) Mix ingredients 11-17 and most of the water together and heat to 80° C.
D) Mix B and C together in a water-jacked mixer
E) Cool down to room temperature with constant mixing to form emulsion
F) Add ingredient 18 in process E
G) Pack the product and store at room temperature for further tests.
   Results of the Tests:

The above-produced blood sugar lowering lotion samples were tested in human to evaluate its functions in real cases (The preclinical evaluations were conducted by doctors in China). In real testing cases, about 1 gram of the blood sugar lowering lotion produced above was applied to the arm of patient and blood sugar levels monitored by doctors. Results of three sets of tests are summarized in Table 1. In all cases, patients had no site effect. In one case, patient's thirsty feeling and other symptoms caused by diabetes disappeared after a week of administration of the invented cream.

TABLE 1
Summary of the test results from three patients*

| Patient | Blood sugar levels | | |
|---|---|---|---|
| # | Before use | After use | Comments |
| A | 13.89 | Normal | Newly diagnosed patient, age 46, feel |
| | | | normal after using the invented lotion |
| | | | for one week (all symptoms related to |
| | | | diabetics disappeared). |
| B | >9.2 | 8.1 | Patient diagnosed with diabetics for 4 |
| | | | years. Could not control blood sugar |
| | | | level via dieting. |
| C | 10.18 | 6.49 | Patient retired, blood sugar level back to |
| | | | 6.49 after one week of use of the |
| | | | invented lotion. |

*All data presented in the table were collected before eating.

1. An herb extract based cosmeceutical cream product for controlling the blood sugar levels of non-insulin dependent diabetes (type II diabetes) comprising hypocaemics ingredients of anti-diabetic herb extracts, a skin care cream base, and components of hypocaemics drugs and solvents for dissolving them.

2. A process for making the herb extract based hypocaemics cream, which comprises:
A) Making hypocaemics ingredients of anti-diabetic herb extracts;
B) Dissolving all water soluble ingredients in a hydrophilic aqueous system;
C) Dissolving all water insoluble ingredients in a hydrophobic solvent system;
D) Mixing the hydrophilic aqueous system with the hydrophobic solvent system to make a hydrophilic/hydrophobic mix;
E) Cooling down the mixed system and homogenizing the mix to get final stable cream product.

3. An herb extract based cosmeceutical cream product as claimed in claim 1, wherein the external skin-care cream is in the lotion (or cream) type form having the function of skin care and lowering blood sugar levels of non-insulin dependent diabetes.

4. An herb extract based cosmeceutical cream product as claimed in claim 1, wherein the external skin care cream is in the lotion type form having the function of lowering blood sugar levels of non-insulin dependent diabetes (type II diabetes).

5. An herb extract based cosmeceutical cream product as claimed in claim 1, wherein the hypocaemics ingredients of herb extracts is selected from the group of traditional anti-diabetic medicines consisting of Vaccinium myrtillus, Crataegus oxyacantha/C. monogyna, Baical Skullcap, Ginkgo biloba, Gymnema sylvestra, Fenugreek, Galega officinalis, and onion.

6. An herb extract based cosmeceutical cream product as claimed in claim 1, wherein the hypocaemics ingredients of herb extracts is selected from the group of traditional anti-diabetic medicines consisting of Vaccinium myrtillus, Crataegus oxyacantha/C. monogyna, Baical Skullcap, Ginkgo biloba, Gymnema sylvestra, Fenugreek, Galega officinalis, and onion. The herb extract can be used individually or in combination (a mix of them).

7. The herb extract based hypocaemics ingredients of herb extracts as claimed in claim 2, wherein the traditional anti-diabetic herb medicine ingredient subject to extraction is a mix of water soluble and solvent extracted parts of a subject herb medicine.

8. The herb extract based hypocaemics ingredients of herb extracts as claimed in claim 2, wherein the active hypocaemic components are water-soluble.

9. An herb extract based cosmeceutical cream product as claimed in claim 1, wherein the skin care cream base can be selected from a wide arrange of formula combinations.

10. An herb extract based cosmeceutical cream product as claimed in claim 2, wherein amount of hypocaemics ingredients of herb extracts formulated in the composition is 0.5% to 30% by weight or more, based upon the concentration of active compounds in the extract and type of extracts.

11. An herb extract based cosmeceutical cream product as claimed in claim 6, wherein the amount of hypocaemics ingredients of herb extracts formulated in the composition is 10% to 30% by weight or more when the extract is non-concentrated.

12. A cosmeceutical cream product as claimed in claim 1, wherein the blood sugar lowering product containing other known elements of anti-diabetic drug compositions including chlorpropamide, glibenclamide, gliclazide, glimepiride, glipizide, tolbutamide, glycyrrhizic acid (salt), glycyrrhetic acid, nicotinic amide, pantothenyl ethyl ether, allantoin, hinokitiol, bisabold, eucalyptone, thymol, inositol, tranexamic acid, arbutin, cepharanthine, saphora flavescens, malva sylvestris, and thiazolidinediones.

13. An herb extract based cosmeceutical cream product as claimed in claim 12, wherein the amount of hypocaemics drug components formulated in the composition is 0.5% to 5% by weight.

14. An herb extract based cosmeceutical cream product as claimed in claim 12, wherein the amount of hypocaemics drug components formulated in the composition is 1% to 4% by weight.

15. The method of making an herb extract based anti-diabetic cream as in claim 2, wherein all water soluble ingredients are dissolved in a hydrophilic aqueous system, and heated to 50-100° C. for 1 to 10 minutes, the preferred heating condition is 60-90° C. for 1-5 minutes.

16. The method of making an herb extract based anti-diabetic cream as in claim 2, wherein all water insoluble ingredients are dissolved in a hydrophobic solvent system, and heated to 50-100° C. for 1 to 8 minutes.

17. The method of making an herb extract based anti-diabetic cream as in claim 2, wherein all water insoluble ingredients are dissolved in a hydrophobic solvent system, and heated to 60-90° C. for 1 to 5 minutes.

18. The method of making an herb extract based anti-diabetic cream as in claim 2, wherein a hydrophilic aqueous system and a hydrophobic solvent system prepared is well mixed to make a hydrophilic/hydrophobic emulsion.

19. The method of making an herb extract based anti-diabetic cream as in claim 2, wherein the mixed system is subjected to cool down and homogenized to get final stable cream product.
